(19) United States
(12) Patent Application Publication
(10) Pub. No.: US 2005/0209243 A1
(43) Pub. Date: Sep. 22, 2005

(54) Theophylline-based soluble guanylyl cyclase activators KMUP-1 analogues enhanced cyclic GMP and K+ channel activities on rabbit corpus cavernosum smooth muscle and intercavernous pressure activities

(75) Inventor: Ing-Jun Chen, Kaohsiung (TW)

Correspondence Address: BIRCH STEWART KOLASCH & BIRCH, PO BOX 747, FALLS CHURCH, VA, 22040-0747, US

(73) Assignees: Ing-Jun Chen; Cho-Jan Liang

(21) Appl. No.: 11/134,293

(22) Filed: May 23, 2005

Related U.S. Application Data
(63) Continuation-in-part of application No. 10/256,115, filed on Sep. 27, 2002.

(51) Int. Cl. A61K 31/522; C07D 473/04

(52) U.S. Cl. 514/252.16; 544/270

(57) ABSTRACT

The present invention provides the pharmaceutical compositions for relaxing corpus the cavernosal smooth muscle, increasing the intracarvernosal pressure, and enhancing learning and memory activities.

### FIELD OF THE INVENTION

The present invention relates to compounds of theophylline based KMUP-1 and KMUP-2 (KMUPs) performing actions on the endothelium-dependent NO releasing, the soluble guanynyl cyclase (sGC) activation, the minimum phosphodiesterase (PDE) inhibition, the K+-channel opening, the relaxation of corpus carvernosal smooth muscle, the increase of intracarvernosal pressure (ΔICP), and enhanced learning and memory activities.

### BACKGROUND OF THE INVENTION

Inhibition of type 4 phosphodiesterase (PDE) by xanthine derivatives, including methylxanthine, such as theophylline, usually increases intracellular formation of cyclic AMP in various smooth muscle cells. Among them, theophylline is tranditionaly a non-selective PDE (phosphodiesterase) inhibitor (Beavo et al. Physiol Rev., 75, 725-748, 1995).

KMUP-1, a theophylline-based derivative, has been described to have not only minimum PDE (phosphodiesterase) inhibition but also enhanced cyclic GMP (guanosine 3′,5′-cyclic monophosphate) increasing activities in the previous report (WU, B. N. et al. Br. J. Pharmacol., 134, 265-274, 2001). Type 5 PDE (phosphodiesterase) inhibitor such as sildanafil, with cyclic GMP (guanosine 3′,5′-cyclic monophosphate) increasing activity, has been proved to be effective in the treatment of penile dysfunction after its oral administration in man (Goldstein I. et al. N. Engl. J. Med., 338, 1397-1404, 1998). However, KMUP-1, with minimum PDE (phosphodiesterase) inhibition as YC-1 but also with NO-releasing, and NO-independent sGC activation activity, similar to YC-1, to increase cyclic GMP activities in rat aortic smooth muscle, was thus supposed similarly to have rabbit cavernosal smooth muscle relaxation and penile erection effects in this invention.

Activation of soluble guanyl cyclase (sGC) induces the formation of cyclic GMP (guanosine 3′,5′-cyclic monophosphate), which is a second messenger of NO action, generally modulates the activity of its effector proteins that lead to vasorelaxation (Schmidt T. et al. Biochim. Biophys. Acta., 1178, 153-175, 1993) and also the opening of K⁺-channel (Murphy M. E. and Brayden, J. E. J. Physiol., 489, 723-734, 1995). It is reasonable to suggest that activation of sGC (soluble guanylyl cyclase) and inhibition of phosphodiesterase (PDE) that metabolize the cyclic GMP (guanosine 3′,5′-cyclic monophosphate), may together attribute to the increase of cyclic GMP associated vascular and cavernosal smooth muscle relaxations. YC-1 is a representative of this class of NO (nitric oxide)-independent sGC activator with PDE inhibition and may lead to a long-lasting cyclic GMP-mediated inhibition of vasoconstriction (Wu C. C. et al. Br. J. Pharmacol., 116, 1973-1978, 1995; Galle J. et al., Br. J. Pharmacol., 127, 195-203, 1999). Recently, BAY 41-2272 and BAY 51-9491 potently activate sGC (soluble guanylyl cyclase) by a mechanism that is also NO-independently (Stasch et al. Nature., 410, 212-215, 2001). KMUP-1 is thus suggested to have YC-1 like activity but having the different chemical structure, characteristicly with theophylline base.

To date, sildanafil has been described to abolish vas deferens contractility initiated by K⁺ channel blocker (Medina et al. Br. J. Pharmacol., 131, 871-874., 2000); YC-1 mediate stimulation of Ca²⁺-I_{K(ca)} and effectively inhibited the voltage-dependent K⁺ current I_{K(V)} in GH₃ lactotrophs (Wu et al Neuropharmacology., 39, 1788-1799., 2000). In contrast, KMUP-1 not only displayed minimum inhibition of PDE (phosphodiesterase) and enhanced activation of sGC (soluble guanylyl cyclase), but also reversed the K⁺ channel blockade caused by K⁺ channel blockers in rat aortic smooth muscle (Wu et al., Br. J. Pharmacol., 134, 265-274.2001).

K⁺ channel openers, acting by liberation of NO (nitric oxide), have been shown to relax human isolated CCSMs (corpus cavernosum smooth muscle) and produce erection when injected intracorporeally into animals and men (Saito et al Urol. Res., 26, 137-141, 1998). Many experimental animals have been employed in in vivo studies of penile erection (Rehman J. et al., Urology., 51, 640-644, 1998; Seyam R. M. et al., Urology., 50, 994-998, 1997; Trigo-Rocha et al., 1993; Lue, T. F. Semin. Urol., 4, 217-224, 1986; Wang R. et al., J. Urol., 151, 234-237, 1994; Stief C. G. et al., J. Urol., 159, 1390-1393, 1998). KMUP-1, a methyl xanthine and piperazine derivative, structurally with 6 nitrogen atoms as sildanafil and with an ethylpiperazine moiety on the position 7 of theophylline-base, combining the PDE (phosphodiesterase) inhibition, sGC (soluble guanylyl cyclase) stimulation, and K⁺ channels opening activity, has been described to achieve the full relaxation activity in rat aortic smooth muscle (Wu B. N. et al., Br. J Pharmacol., 134, 265-274, 2001). Taking above consideration, we are thus encouraged to use KMUP-1, multiply with above effects in vasculature, to examine its possible effects in rabbit CCSM and penile erection, including associated K⁺-channel opening activity.

In this invention, we characterized the effects of KMUP-1 on rabbit CCSMs (corpus cavernosum smooth muscles) and associated NO/sGC/cGMP activation, retard of induced K⁺ channels blocking and PDE (phosphodiesterase) inhibiting activities. It is to be noted in erectile dysfunction that associated with vascular relaxation, the combination use of PDE inhibitor and sGC (soluble guanylyl cyclase) stimulator or K⁺-channel opener was suggested to enhance the results achieved (Notable Martinez-Pineiro et al. Arch. Esp. Urol., 49, 270-276., 1996). The present invention provides KMUP-1, with those possible activities and intracavernous pressure (ICP) increasing effect for the management of erectile dysfunction.

### SUMMARY OF THE INVENTION

This invention relates to KMUP-1 and KMUP-2, which upon laboratory testing on animals have been proven that they pharmacologically possesses NO releasing from endothelium, sGC activation on smooth muscle, minimum type Five phosphodiesterase inhibition, relaxation of corpus carvernosal smooth muscle and increase of intracarvernosal pressure (ΔICP). Relaxation of KMUP-1 and KMUP-2 was attenuated by endothelium removed, high K⁺ and pretreatments with soluble guanylyl cyclase (sGC) inhibitors ODQ, a NOS inhibitor L-NAME, a K⁺ channel blocker TEA, a K_{ATP} channel blocker glibenclamide, a voltage-dependent K⁺ channel blocker 4-AP and Ca²⁺-dependent K⁺ channel blockers apamin and charybdotoxin. The relaxant responses of KMUP-1 together with a standard PDE (phosphodiesterase) inhibitor IBMX (3-isobutyl, 1-methyl xanthine) had additive actions on rabbit corpus cavernosum smooth muscle.

The above aspects and advantages of the present invention will become more readily apparent to those ordinarily skilled in the art after reviewing the following detailed description and accompanying drawings, in which:

### BRIEF DESCRIPTION OF THE DRAWINGS

FIG. 1 shows the chemical structures of KMUP-1, KMUP-2 and sildanafil;

FIG. 2 shows the effects of KMUP-1 on phenylephrine (10 μM)-precontracted rabbit corpus cavernosum in the endothelium-denuded EC (−) and endothelium-intact EC (+) corporeal smooth muscle strips. *P<0.05, n=12 as compared with the KMUP-1 (two way repeated measures ANOVA followed by Student-Newman-Keuls test). A . . . EC (+) B . . . EC (−)

FIG. 3 shows the effects of KMUP-1 on the rabbit corpus cavernosum, precontracted with phenylephrine (10 μM) and 60 mM KCl, respectively. *P<0.05, **P<0.01, ***P<0.001, n=12 as compared with the KMUP-1 (two way repeated measures ANOVA followed by Student-Newman-Keuls test). A . . . Phenylephrine (10 μM) B . . . KCl (60 mM)

FIG. 4(A) shows the effects of KMUP-1 on phenylephrine (10 μM)-precontracted rabbit corpus cavernosum in the absence and presence of potassium channel blockers. *P<0.05, n=12 as compared with the KMUP-1 (two way repeated measures ANOVA followed by Student-Newman-Keuls test). *P50.05, n=12 as compared with the KMUP-1 (two way repeated measures ANOVA followed by Student-Newman-Keuls test).

FIG. 4(B) shows the effects of sildanafil on phenylephrine (10 μM)-precontracted rabbit corpus cavernosum in the absence and presence of potassium channel blockers. *P<0.05, n=12 as compared with the KMUP-1. A . . . control; B . . . after TEA (10 mM); C . . . after glibenclamide (1 μM); D . . . after 4-AP (100 μM); E . . . after apamin (1 μM); F . . . after charybdotoxin (ChTX, 0.1 μM).

FIG. 5(A) shows the effects of KMUP-1 on phenylephrine (10 μM)-precontracted rabbit corpus cavernosum in the absence and presence of L-NAME (100 μM), ODQ (1 μM). *P<0.05, n=12 as compared with the KMUP-1 (two way repeated measures ANOVA followed by Student-Newman-Keuls test).

FIG. 5(B) shows the effects of sildanafil on phenylephrine (10 μM)-precontracted rabbit corpus cavernosum in the absence and presence of L-NAME (100 μM), ODQ (1 μM). *P<0.05, n=12 as compared with the KMUP-1 (two way repeated measures ANOVA followed by Student-Newman-Keuls test). A . . . control; B . . . after L-NAME (100 μM); C . . . after ODQ(1 μM)

FIG. 6(A) shows the additive effects of KMUP-1, KMUP-2 and IBMX on phenylephrine-precontracted rabbit carvernosal strips. Each value represents the mean ±S.E., *P<0.05, n=8 as compared with the control value. (ANOVA followed by Dunnett's test). Control: solvent control. 1 . . . Vehicle; 2 . . . IBMX (0.5 μM); 3 . . . KMUP-1 (0.01 μM); 4 . . . IBMX (0.5 μM)+KMUP-1 (0.01 μM); 5 . . . KMUP-2 (0.05 μM); 6 . . . IBMX (0.5 μM)+KMUP-1 (0.05 μM); 7 . . . KMUP-2 (0.1 μM); 8 . . . IBMX (0.5 μM)+KMUP-1 (0.1 μM)

FIG. 6(B) shows the additive effects of KMUP-2 (0.01, 0.05, 0.1 mM) and IBMX (0.5 mM) on phenylephrine (10 mM)-precontracted rabbit aortic rings.

FIG. 6(C) shows the additive effects of KMUP-2 (0.01, 0.05, 0.1 mM) and IBMX (0.5 mM) on phenylephrine (10 mM)-precontracted corpus cavernosum. Each value represent the mean ±S.E., *P<0.05, n=8 as compared with the control value (ANOVA followed by Dunnett's test). 1: Vehicle; 2: IBMX (0.5 μM); 3: KMUP-2 (0.01 μM); 4: IBMX (0.5 μM)+KMUP-2 (0.01 μM); 5: KMUP-2 (0.05 μM); 6: IBMX (0.5 μM)+KMUP-2 (0.05 μM); 7: KMUP-2 (0.1 μM); 8: IBMX (0.5 μM)+KMUP-2 (0.1 μM).

FIG. 7(A) shows the effects of KMUP-1 (0.01, 0.1, 1, 10 m M) and sildanafil (0.01, 0.1, 1, 10 m M) on guanosine 3′,5′-cyclic monophosphate levels in rabbit corpus cavernosum smooth muscle cells;

FIG. 7(B) shows the effects of KMUP-2 (0.1, 1, 10, 100 mM) and sildanafil (0.1, 1, 10, 100 mM) on guanosine 3′,5′-cyclic monophosphate levels in rabbit aorta smooth muscle cells;

FIG. 7(C) shows the effects of KMUP-2 (0.1, 1, 10, 100 mM) and sildanafil (0.1, 1, 10, 100 mM) on guanosine 3′,5′-cyclic monophosphate levels in rabbit corpus cavernosum smooth muscle cells, 1:. KMUP-1; 2:. sildanafil; and 3: KMUP-2.

FIG. 8(A) shows the effects of KMUP-1 (10 μM) on guanosine 3′,5′-cyclic monophosphate levels in rabbit corpus cavernosal smooth muscle cells and in the absence or presence of ODQ (10 μM) and methylene blue (100 μM). Each value represents the mean ± S.E., from 3 independent experiments. **P<0.01 as compared with the KMUP-1 (ANOVA followed by Dunnett's test), 1: KMUP-1; 2: after ODQ.

FIG. 8(B) shows the effects of KMUP-2 on guanosine 3′,5′-cyclic monophosphate levels in rabbit aorta smooth muscle cells in the absence or presence of ODQ (10 mM) and methylene blue (100 mM), 1: KMUP-2; 2: after methylene blue; 3: after ODQ.

FIG. 8(C) shows the effects of KMUP-2 on guanosine 3′,5′-cyclic monophosphate levels in corpus cavernosum smooth muscle cells and in the absence or presence of ODQ(10 mM) and methylene blue (100 mM), 1: KMUP-2; 2: after methylene blue; 3: after ODQ.

FIG. 9 shows the synthesis scheme of the compound A.

### DETAILED DESCRIPTION OF THE PREFERRED EMBODIMENT

The invention is described more specifically with reference to the following embodiments. It is to be noted that the following descriptions of preferred embodiments of this invention are presented herein for the purpose of illustration and description only; it is not intended to be exhaustive or to be limited to the precise form disclosed.

The present provides serial theophylline (1-methyl, 3-methylxanthine) and 1-methyl, 3-isobutyl xanthine (IBMX) derivatives chemically with formula I and II:

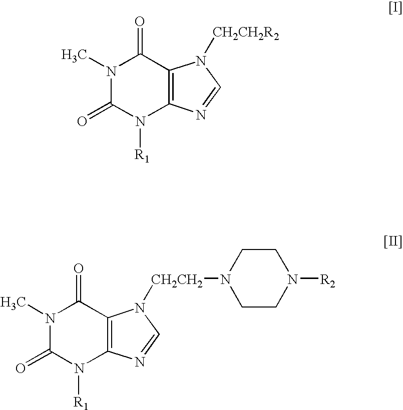

R₁ is —CH₃ or —CH₂CH (CH₃)₂; R₂ is 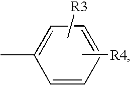
wherein R₃ and R₄ are selected from the group consisting of OCH₃, CH₃, halogen, and NO₂, and the halogen is one of F, Cl, Br and I.

The nonadrenergic/noncholinergic neurotransmitter NO (nitric oxide) plays a crucial role in attenuating smooth muscle contraction, inducing smooth muscle relaxation and penile erection. Several vasoactive agents, including NO and PDE (phosphodiesterase) inhibitors, initiate and/or enhance CCSM (corpus cavernosum smooth muscle) relaxation (Soderling S. H. et al., Curr. Opin. Cell Biol., 12, 174-179, 2000). Soluble guanylyl cyclase, when activated by NO, catalyzes the formation of cGMP from GTP, whereas cGMP-specific phosphodiesterases (PDEs) catalyze the hydrolysis of cGMP to GMP. Termination of signal transduction by hydrolysis of cGMP (guanosine 3′,5′-cyclic monophosphate) depends on the specificity and expression of PDE (phosphodiesterase) isozymes in the target tissues (Juilfs et al., 1999). One such class of drugs is sildanafil, an inhibitor of cyclic GMP-specific PDE, for use in male erectile dysfunction (Wallis R. M. et al., Am. J. Cardiol., 83, 3C-12C, 1999).

The physiologic regulation of penile tumescence involves a balance between relaxant and contractile events. Relaxation is mainly promoted by endothelium-dependent mechanisms and stimulation of nitrergic nerves. In contrast, the adrenergic neuro-transmission has been reported as a promoter of penile flaccidity through the activation of alpha-adrenergic receptors (Angulo J. et al., Urology., 57, 585-589, 2001).

It is to be emphasized that KMUPs compounds including KMUP-1 and KMUP-2 are classified into derivatives of theophylline-based compounds. The physicochemical Data of N7-substituted theophylline, KMUP-1 and KMUP-2, are illustrated in Table I.

| | MS(Scan | | |
|---|---|---|---|
| Compound | FAB+) | ¹H-NMR(CDCl₃) | |
| 1 | 402.88 | δ: | 3.60(s, 3H, NCH₃), 3.42(s, 3H, NCH₃) |
| (KMUP-1) | | | 4.45(t, 2H, NCH₂), 2.85(t, 2H, NCH₂) |
| | | | 2.70(t, 4H, 2×CH₂), 3.04(t, 4H, 2×CH₂) |
| | | | 6.97-7.01(m, 2H, 2×Ar—H), |
| | | | 7.27-7.36(m, 2H, 2×Ar—H), |
| | | | 7.69(s, 1H, imidazole-H) |
| 8 | 398.46 | δ: | 3.42(s, 3H, NCH₃), 3.61(s, 3H, NCH₃) |
| (KMUP-2) | | | 2.89(t, 2H, NCH₂), 4.49(t, 2H, NCH₂) |
| | | | 2.75(t, 4H, 2×CH₂), 3.09(t, 4H, 2×CH₂) |
| | | | 3.86(s, 3H, OCH₃), |
| | | | 6.88-7.06(m, 4H, 4×Ar—H) |
| | | | 7.72(s, 1H, imidazole-H) |

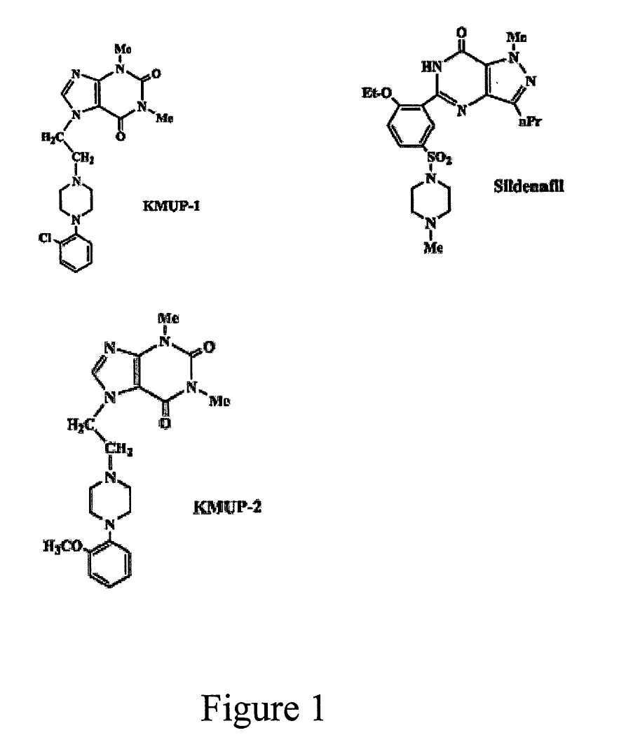

The KMUP-1 and KMUP-2, theophylline-based derivatives, are shown in FIG. 1. KMUP-1 is 7-[2-[4-(2-chlorophenyl)piperazinyl]-ethyl]-1,3-dimethylxanthine), and KMUP-2 is 7-[2-[4-(2-methoxyphenyl)piperazinyl]-ethyl]-1,3-dimethylxanthine). The derivatives of theophylline-based compounds, for example shown as aforesaid formula I and II, wherein R₁ is —CH₃ or —CH₂CH (CH₃)₂; R₂ is 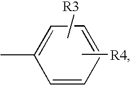
in which R₃ and R₄ are selected from the group of OCH₃, halogen and NO₂, and the halogen is one of F, Cl, Br and I.

General synthesis of theophylline-based analogues was performed, and the compounds A and B were obtained by the following step. The compound A or B was dissolved in methanol, according to targeting products required, and added with suitable reagent to obtain the derivative compound of theophylline. The theophylline (1,3-dimethylxanthine) was dissolved in dibromoethane in a glass reacter, boiled and mixed at 100° C. on mantle heater and refluxed for 4 hours, equipped with a cooling condenser to return the vaporized solvent. Till solid completely melt, the reaction was added with NaOH as catalyst to react under 100° C. and boiled for overnight to form the white solid precipitated and NaBr soluble in upper water layer. The white solid precipitate was filtered, dissolved in methanol, concentrated under reduced pressure to obtain white coarse crystal, and then re-crystallized with methanol to obtain the pure white crystal compound A (N7-bromoethyl theophylline).

3-isobutyl-1-methylxanthine (IBMX) was dissolved into dibromoethane in a glass reacter, boiled and mixed at 100° C. on mantle heater and refluxed for 4 hours, equipped with a cooling condenser to return the vaporized solvent. Till solid completely melt, the reaction was added with NaOH as catalyst to react under 100° C. and boiled for overnight to form the white solid precipitated and NaBr soluble in upper water layer. The white solid precipitate was filtered, dissolved in methanol, concentrated under reduced pressure to obtain white coarse crystal, and then re-crystallized with methanol to obtain the pure white crystal compound B (N7-bromoethyl IBMX).

The compound A dissolved in methanol, according to targeting products required, was added with 1-(2-Chlorophenyl)piperazine, 1-(m-Chlorophenyl) piperazine, 1-(4-Nitrophenyl) piperazine, and 1-(o-Methoxyphenyl) piperazine, respectively, to processes the amination with piperazine moiety, using NaOH as catalyst, and reflux for 4 hours in methanol to be precipitated. The solid product was dissolved and further recrystallized with methanol to obtain theophylline-based or IBMX-based compounds including compounds 1-8 as follows.

| Compound | |
|---|---|
| No. | Name |
| 1 | N7-{2-[4(2-chlorophenyl)piperazinyl]ethyl}, |
| | 1,3-methyl xanthine |
| 2 | N7-{2-[4(3-chlorophenyl)piperazinyl]ethyl}, |
| | 1,3-methyl xanthine |
| 3 | N7-{2-[4-(2-nitrophenyl)piperazinyl]ethyl}, |
| | 1,3-methyl xanthine |
| 4 | N7-{2-[4-(4-nitrophenyl)piperazinyl]ethyl}, |
| | 1,3-methyl xanthine |
| 5 | N7-{2-[4(4-chlorophenyl)piperazinyl]ethyl}, |
| | 1,3-methyl xanthine |
| 6 | N7-{2-[4-(2-nitrophenyl)piperazinyl]ethyl}, |
| | 1,3-methyl xanthine |
| 7 | N7-{2-[4(2-chlorophenyl)piperazinyl]ethyl}, |
| | 3-isobutyl-1-methyl xanthine |
| 8 | N7-{2-[4(2-methoxyphenyl)piperazinyl]ethyl}, |
| | 1,3-methylxanthine |

Similar results were obtained from the reaction of the compound B and above piperazine derivative, in which 1,3-methyl xanthine is substituted with 1-methyl,3-isobutyl xanthine.

It is disclosed in the present invention that KMUP-1 and KMUP-2 (KMUPs) have concentration-dependent relaxant activities in rabbit CCSMs. KMUP-1 and KMUP-2 produced rabbit CCSM (corpus cavernosum smooth muscle) relaxations in both endothelium-intact and deprived muscles. This relaxation of KMUPs was reduced by removing endothelium from CCSMs. Guanethidine and atropine treatment had no significant effects on KMUPs-induced relaxations. These results mean that KMUPs-caused relaxation is un-associated with adrenergic and cholinergic neuronal functions. The relaxant effect of KMUPs on endothelium deprived and NOS (nitric oxide synthase) inhibitor-pretreated CCSMs still exist. It is to be noted that KMUPs might have NO-independent relaxant effects on CCSMs. It was disclosed that NO (nitric oxide) is the major endothelium derived relaxing factor (EDRF) in the penile CCSM (corpus cavernosum smooth muscle) (Kim N. et al., J. Clin. Invest., 88, 112-118, 1991). Relaxation of CCSMs might be formed via NO-elicited activation of gunylate cyclase and cGMP formation (Christ G. J. et al., J. Androl., 14, 319-328, 1993). The CCSM relaxant effects performed by KMUP-1 was significantly blunted but not inhibited by pretreatment with sGC inhibitors and the NOS inhibitor. We suggest that other mechanisms of relaxation are activated in addition to the stimulation of NO/sGC/cGMP pathway.

In the present invention, it is disclosed that the combination of KMUP-1/KMUP-2 (KMUPs) and IBMX have an additive effect on CCSMs relaxations. Recently, we have demonstrated that KMUP-1 affected cGMP breakdown at 100 μM due to the inhibition of the enzyme activity of PDE in human platelets. Furthermore, KMUP-1 significantly raised the intracellular cGMP levels in concentration-dependent manners in primary rabbit CCSM cells. It is confirmed by these results that KMUP-1 activates the NO/sGC/cGMP pathway and inhibits the phosphodiesterase (PDE) or cGMP breakdown, so as to elevate the intracellular cGMP levels leading to the CCSM relaxation as previously in smooth muscle (Wu B. N. et al., Br. J. Pharmacol., 134, 265-274, 2001). K⁺ channel opener reduces the tissue tension or contractile force in response to stimulation of the CCSM (Anderson. K. E. Pharmacol. Rev., 45, 253-308, 1993). Vasodilators depend on the K⁺ channel mechanism lose their effects when exposed to high K⁺ solutions because an increase in extracellular K⁺ attenuates the K⁺ gradient across the plasma membrane, so as to render the K⁺ channel-activating mechanism ineffective (Khan S. A. et al., J. Pharmacol. Exp. Ther., 284, 838-846, 1998). K⁺ channels can regulate corporeal smooth muscle tone, and also play a significant role in corporeal smooth muscle tone (Christ G. J. et al., J. Androl., 14, 319-328, 1993). These authors further suggested that impairment in K⁺ channels activity may contribute to erectile dysfunction. Thus, the possibility of K⁺ channels activation by KMUP-1 was further investigated. The importance of K⁺ channel-mediated hyperpolarization of KMUP-1 was provided by the differential potency of KMUP-1 in relaxing PE-induced versus KCl-induced contractions. KMUP-1 produced relaxation in this way, since its effect was almost completely blunted in high K⁺ (60 mM) condition. In these situations, KMUP-1-induced increase in K⁺ efflux would not hyperpolarize CCSMs (corpus cavernosum smooth muscles) sufficiently to inhibit transmembrane Ca²⁺ influx as in aortic smooth muscle (Wu B. N. et al., Br. J. Pharmacol., 134, 265-274, 2001). Relaxant effects performed by KMUP-1, were reduced by a K⁺ channel blocker TEA (Tetraethylammonium), a K_{ATP} channel blocker glibenclamide (Lee S. W. et al., Int. J. Impot. Res., 11, 179-188, 1999), a voltage-dependent K⁺ channel blocker 4-AP (aminopyridine) (Sobey C. G. et al., Br. J. Pharmacol., 126, 1437-1443, 1999) and Ca²⁺-dependent K⁺ channel blockers apamin (Nakagawa A. et al., J. Cardiovasc. Pharmacol., 14, 38-45. 1989) and ChTX (Charybdotoxin) (Garcia M. L et al., Am. J. Physiol., 269, C1-C10, 1995), so that it is further suggested that the relaxant effect of theophylline-based derivatives, including KMUP-1, might be partly associated with K⁺ channel activities.

In the present invention, intracavernous injection of KMUPs dose-dependently resulted in rises of ICP without significant change in blood pressure. The KMUP and theophylline-based derivatives displayed similar duration of tumescence as sildanafil. The results disclosed here provide the evidence for the previous disclosure (Wu B. N. et al., Br. J. Pharmacol., 134, 265-274, 2001). The CCSM relaxant activities of KMUP-1 are mediated via inhibition of PDE (phosphodiesterase) and associated cGMP metabolism, K⁺ channels activity, and activation of NO/sGC/cGMP pathway. The accumulation of cGMP may further enhance the K⁺ efflux and lead to blunt of Ca²⁺ influx-associated contractility in CCSMs. Combination of these multiple pathways may thus attribute to significant relaxation of CCSMs and associated penile erection. Here, it is suggested that NO-releasing, sGC activation, PDE (phosphodiesterase) inhibition and associated cGMP increasing and K⁺ channel activities of KMUP and theophylline-based derivative are the major determinants for the CCSM (corpus cavernosum smooth muscle) relaxation effects in rabbit. The in vivo results of ICP (intracavernous pressure) for sildanafil and theophylline-based derivative are consistent with the in vitro measurements of cGMP for them. These potent CCSMs relaxant and penile erection activities of theophylline-based derivative might be useful to treat erectile dysfunction.

On the other hands, we also observed that the combination of theophylline-based derivatives has an additive effect to enhance learning and memory in human being. Long-term potentiation (LTP) is a potential cellular mechanism underlying learning and memory. Nitric oxide (NO) acts as a retrograde messenger and its downstream effectors are also involved in the modulation of synaptic plasticity in various brain regions such as hippocampus, amygdala, cerebellum, brain stem, cortex etc. Although nitric oxide (NO) is thought to affect synaptic potentiation in various brain regions, the NO-cGMP-PKG pathway involved in memory acquisition in living animals is still unclear. To address this question, this study investigated the modulation of synaptic plasticity both in vitro and in vivo by using the novel compound KMUP-1, KMUP-2 and associated theophylline-based derivatives, which greatly potentiate the response of soluble guanylate cyclase to NO. Theophylline-based derivative greatly enhanced the induction of LTP in Schaffer collateral-CA1 pathway of hippocampal slices at weak tetanus, which was significantly reduced by L-NAME, ODQ and KT5823. The stimulating parameter which induced long-lasting depression was shifted to a lower frequency by theophylline-based derivative. Furthermore, simultaneous perfusion of theophylline-based derivatives with different concentrations of NO donor induced LTP or LTD when the hippocampal slices were stimulated at a frequency as low as 0.02 Hz. The effects performed by theophylline-based derivative on the behavioral tasks in the following animal models were examined: Morris water maze, passive inhibitory and active avoidance tests, and rotor rod test. It was found that theophylline-based derivative greatly improved learning and memory in these behavioral tasks. KMUP-1 injected (1 mg/kg,i.p.) 10 min before the training shortened the escape latency in water maze, increased the retention scores in passive inhibitory avoidance task, decreased the retention time in active avoidance test, and enhanced the motor coordination in the examination of rotor rod. However, the administration of KMUP-1 for 30 min after foot-shock did not affect the retention of passive inhibitory avoidance. The enhancement of learning behaviors by KMUP-1 and theophylline-based derivative were significantly antagonized by L-NAME and KT5823. KMUP-1 thus enhanced LTP, learning and memory in an NO-cGMP-PKG-dependent pathway and is a promising drug to enhance learning and memory in human being.

The compositions of this invention will include various excipients, carriers or diluents and pharmaceutically approved pH of processed salts in accordance to necessity to form composition with therapeutic efficacy. These pharmaceutical preparations may be in the solid form for oral and rectal administration; the liquid form or non-intestinal injection form; or the ointment form for direct application on affected part. Such solid forms are manufactured according to common pharmaceutical preparation methods, which will include disintegrant like starch; sodium carboxymethyl cellulose, adhesive like ethanol; glycerin, or magnesium stearic acid; lactose to obtain pharmaceutical preparation like tablets or filled into capsules of suppositories. The solution including a compound of this ingredient could use buffers of phosphoric nature to adjust the pH to suitable level, before adding the adjutant; emulsifier to produce injection dose or other liquid preparation. In the present invention, a compound or a pharmaceutical composition could be manufactured by mixing synthetic acid salts with various fundamental preparations to form ointments according to known pharmaceutical manufacturing methods. Pharmaceutical compositions manufactured according to this invention could be used on mammals to produce the efficacy of the main ingredient. General dosage could be adjusted according to the degree of symptoms, and normally a person will require 50 to 300 mg each time, three times per day.

The Pharmacological Activities of the Compounds of the Present Invention Have Been Proven by the Following Pharmacological Experiments.

Male New Zeal and white rabbits (2.5-3 kg) were provided from National Laboratory Animal Breeding and Research Center (Taipei, Taiwan) and housed under conditions of constant temperature and controlled illumination. Food and water were available ad libitum. The study was approved by the Animal Care and Use Committee of the Kaohsiung Medical University. Tetraethylammonium (TEA), 4-aminopyridine (4-AP), glibenclamide, phenylephrine, methylene blue, apamin, charybdotoxin, 1H-[1,2,4]Oxadiazolo[4,3-a]quinoxalin-1-one (ODQ), N^{w}-nitro-L-arginine methyl ester (L-NAME) and 3-isobutyl-1-methylxanthine (IBMX) were obtained from Sigma Chemical Co. (St. Louis, Mo., U.S.A.). Sildanafil citrate was supplied by the Cadila Healthcare Ltd. (Maninagar, India). All other reagents used were from E. Merck (Darmstadt, Germany). KMUP-1 provided y the present invention was dissolved in 10% absolute alcohol, 10% propylene glycol and 2% 1N HCl at 10 mM. Serial dilutions were made in distilled water.

Corpus Cavernosum Smooth Muscle Relaxant Activity

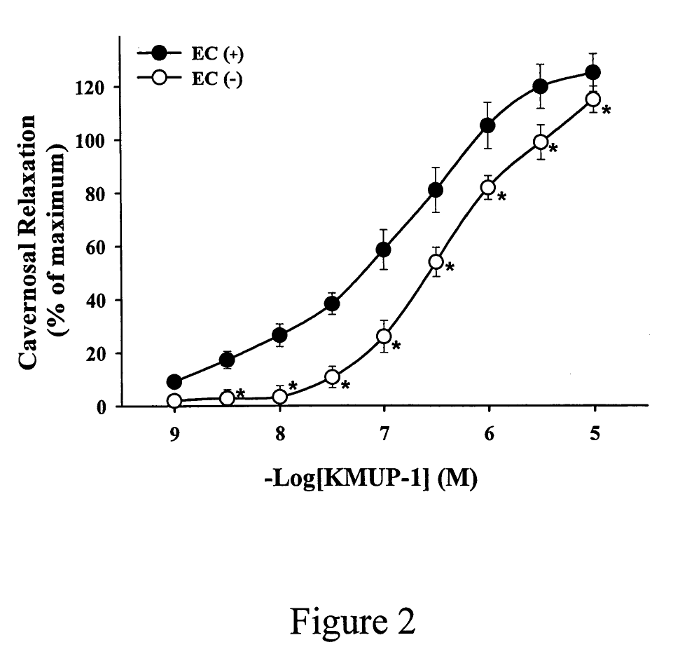

As shown in FIG. 2, cumulative concentrations of KMUP-1 (0.001-10 μM) produced concentration-dependent relaxations both in endothelium-denuded (EC−) and endothelium-intact (EC+) corpus cavernosum smooth muscles. It means that KMUP-1 induced endothelium-independent relaxation. However, KMUP-1 indeed has a significant shift in the response curve after endothelium denudation. It means that at least part of the observed effect is endothelium-dependent. The estimated EC₅₀ value for KMUP-1 in EC+ corpus cavernosum smooth muscles was −log EC₅₀ =7.19±0.09. Additionally, KMUP-1 completely relaxed the corpus cavernosum smooth muscle strips at 10 μM.

Effects on K⁺ Channels

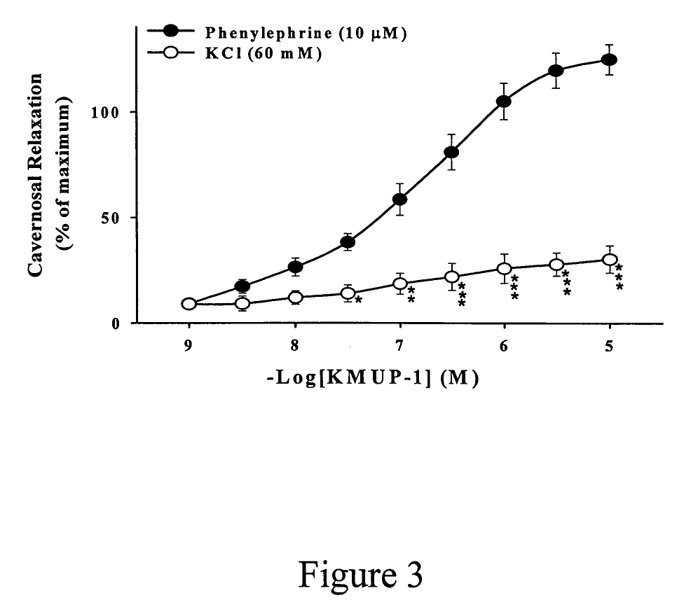

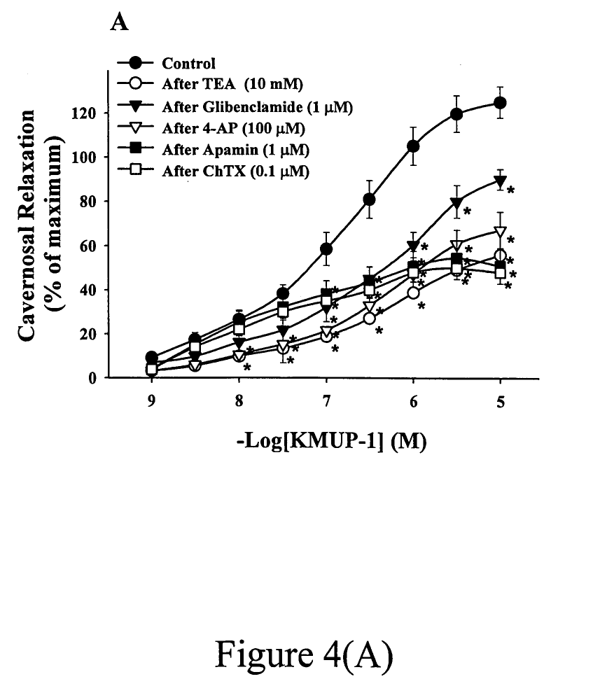

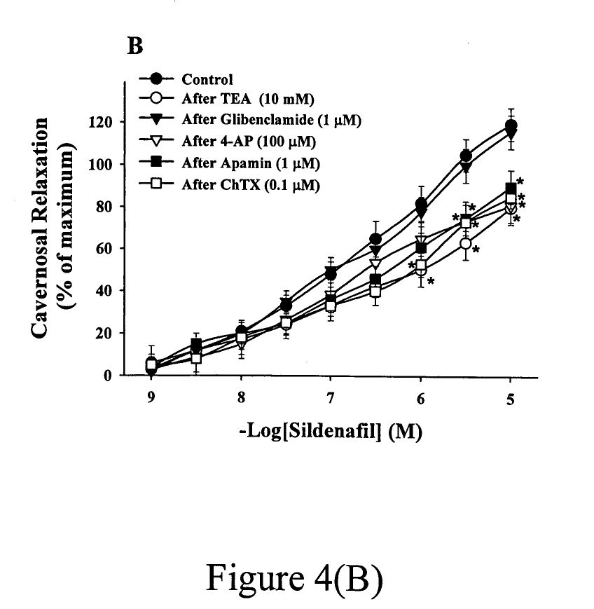

KMUP-1 (0.001-10 μM) caused a concentration-dependent relaxation in phenylephrine-contracted corpus cavernosum smooth muscles. However, KMUP-1 had a great reduction of relaxation in the presence of high K⁺ (60 mM) as shown in FIG. 3. The corpus cavernosum smooth muscles relaxation of KMUP-1 was inhibited by a K⁺ channel blocker Tetraethylammonium (TEA) (−log EC₅₀=5.37±0.05), a K_{ATP} channel blocker glibenclamide (−log EC₅₀=6.57±0.15), a voltage-dependent K⁺ channel blocker 4-aminopyridine (4-AP) (−log EC₅₀=5.83±0.17) and Ca²⁺-dpendent K⁺ channel blockers apamin (−log EC₅₀=5.85±0.11) and charybdotoxin (−log EC₅₀=5.63±0.09) as shown in FIG. 4.

Effects on NO Synthase and Soluble Guanylyl Cyclase

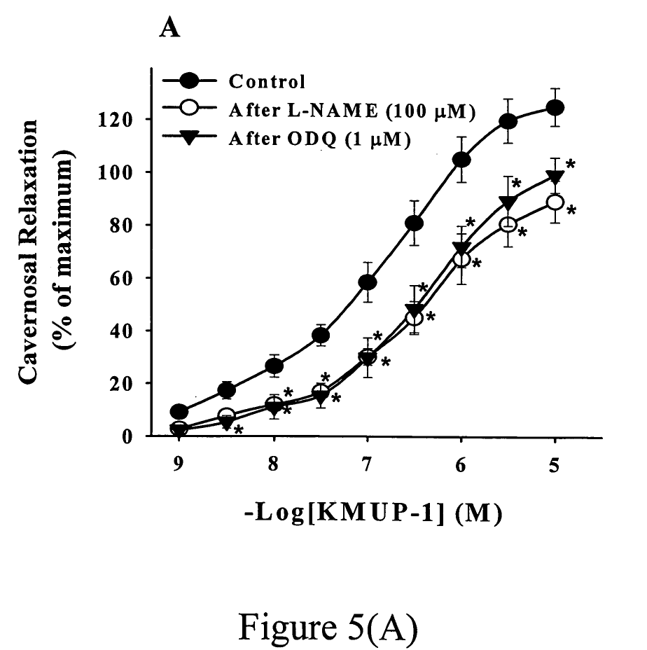

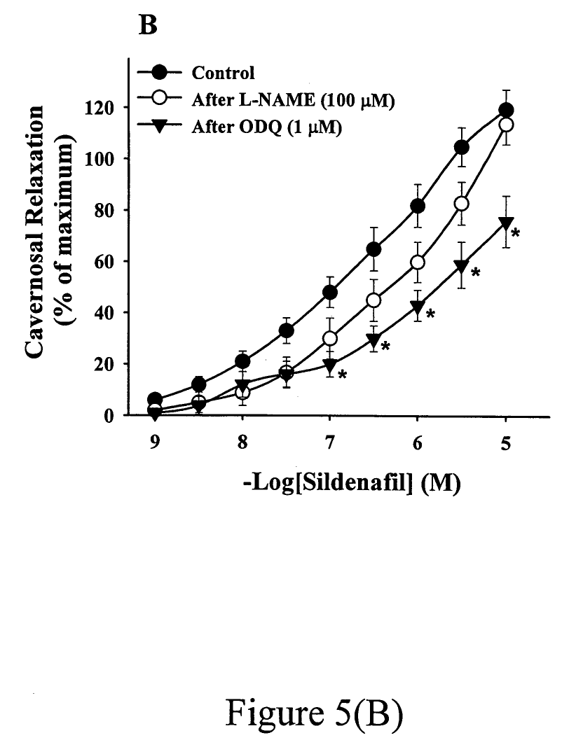

Pretreatment with a NOS (nitric oxide synthase) inhibitor L-NAME (−log EC50=6.51±0.08) and sGC (soluble guanylyl cyclase) inhibitors ODQ (1H-[1,2,4]Oxadiazolo [4,3-a]quinoxalin-1-one) (−log EC50=6.79±0.12), the relaxations elicited by KMUP-1 (−log EC50=7.19±0.09) were significantly inhibited as shown in FIG. 5.

Phosphodiesterase Assay

Human platelets were isolated from whole blood by using 50 mM Tris-HCl with 5 mM MgCl₂ (pH 7.5) and centrifuged to prepare platelet rich plasma. Washed human platelets were resuspended in 50 mM Tris-HCl with 5 mM MgCl₂ (pH 7.5). Platelets were then disrupted by sonication, and soluble PDE preparation was obtained by ultracentrifugation 105,000 g at 4° C. for 60 min. The enzyme (11.5 mg/10 μl) was incubated with Tris-HCl (80 μl) and 10 μM cyclic GMP substrate (final concentration 1 μM with 0.1 μCi [³H]-cyclic GMP) was added. After 20 min at 37° C., the samples were heated to 100° C. for 2 min. Ophiophagus Hannah snake venom (10 mg/ml, 10 μl) was then added and incubated at 37° C. for 10 min to convert the 5-GMP to the unchanged nucleosides, guanosine. An ion-exchange resin (200 μl) was added to bind all uncoverted cyclic GMP. After centrifuging, the supernatant was removed for determination in a liquid scintillation counter.

Inhibition of Phosphodiesterase Activity

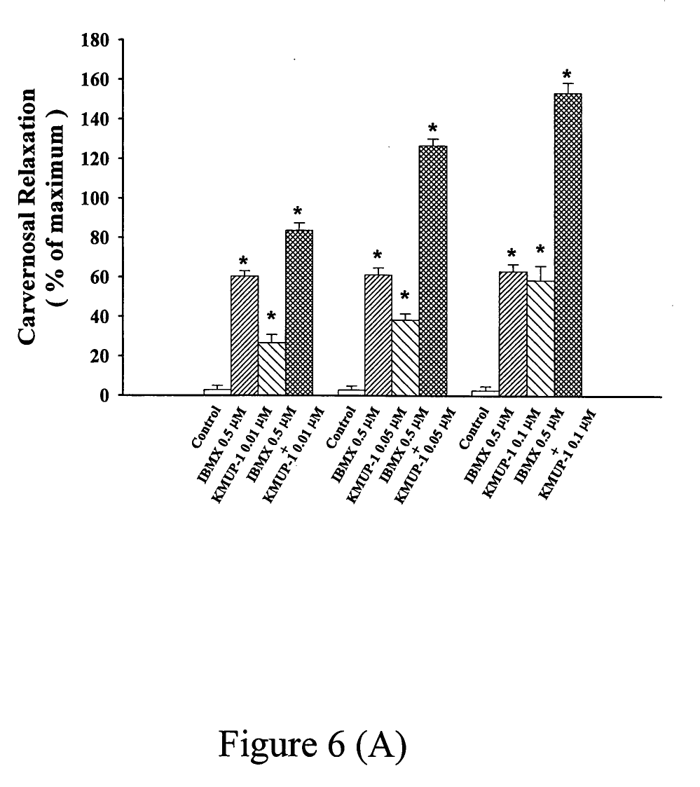

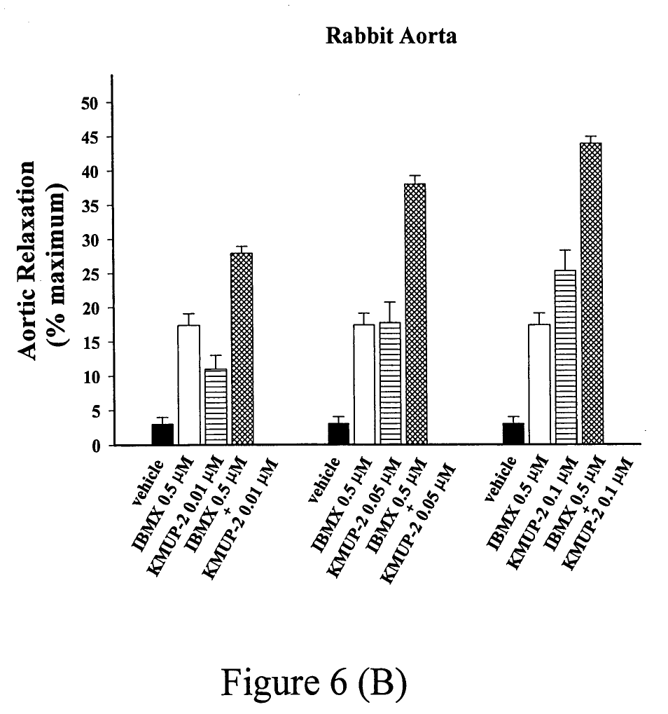

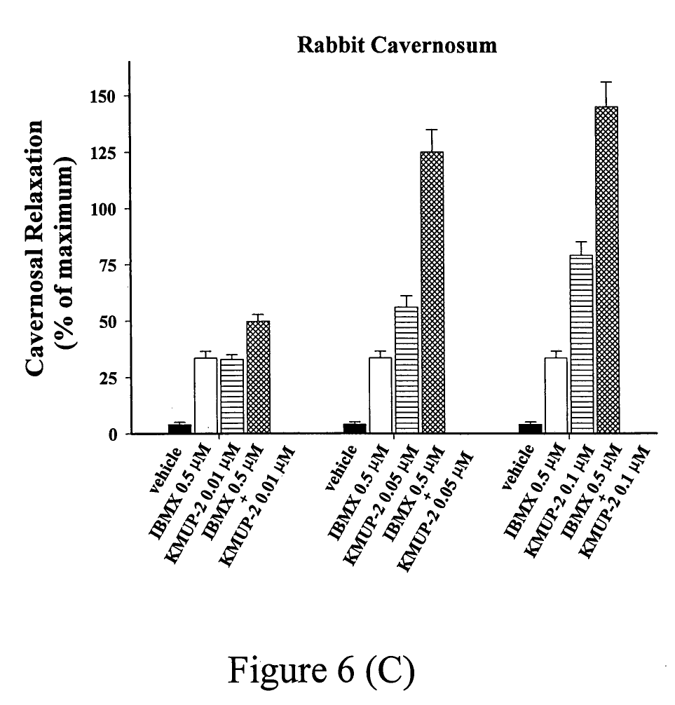

Effects performed by KMUP-1 on corpus cavernosum smooth muscles were investigated after inhibition of PDE (phosphodiesterase) activity by IBMX. KMUP-1 (−log EC₅₀=7.21±0.12) and IBMX (0.5 μM) were additively (−log EC₅₀=8.65±0.14) to induce relaxation (FIG. 6A). KMUP-1 (0.01, 0.05, 0.1 μM)-induced vasorelaxation (−log EC50=7.03 0.10) had an additive effect in the presence of IBMX (0.5 μM) (−log EC50=10.60 0.13). In further experiments, KMUP-1 inhibited the PDE activity (29±3.1%, n=3, each performed in triplicate) at 100 μM while in comparison with sildanafil (94±4.8%, n=3) (Bin-Nan Wu et al., Br J Pharmacol, 134, 265-274, 2001).

The effects performed by KMUP-2 on aortic rings and corpus cavernosum smooth muscle were investigated after inhibition of PDE (phosphodiesterase) activity by using IBMX, a nonselective PDE (phosphodiesterase) inhibitor. KMUP-2 (0.01, 0.05, 0.1 μM)-induced relaxation in aortic rings (−log EC₅₀=5.69±0.02) and in cavernosum smooth muscle (−log EC₅₀=7.58±0.02) had an additive effect in the presence of IBMX (0.5 μM, −log EC₅₀=6.61±0.12 and −log EC₅₀=8.37±0.03, respectively) as shown in FIG. 6B. In further experiments, KMUP-2 inhibited the PDE (phosphodiesterase) activity (21±2.1%, n=3, each performed in triplicate) at 100 μM in comparison with sildanafil (94±4.8%, n=3) (Rong-lyh Lin et al., Drug Development Research 55, 162-172, 2002).

Guanosine 3′,5′-Cyclic Monophosphate Enhancing Activity in Cavernosum Smooth Muscle Cells

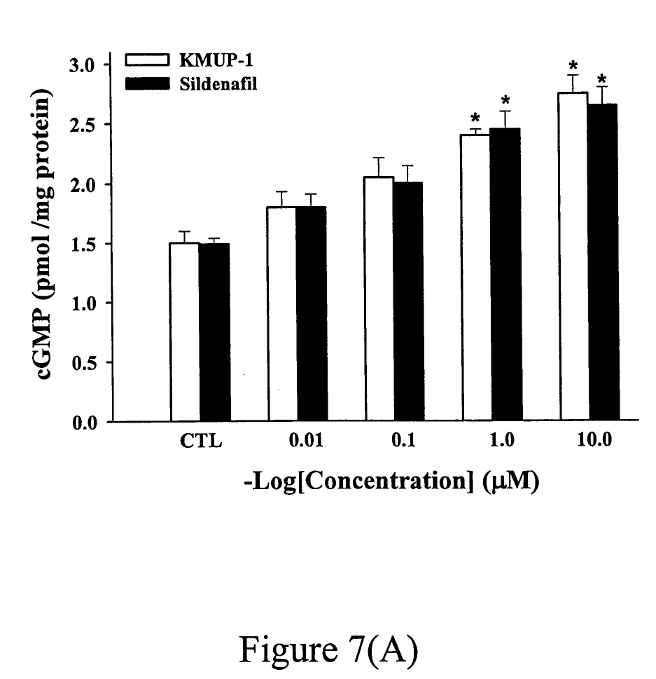

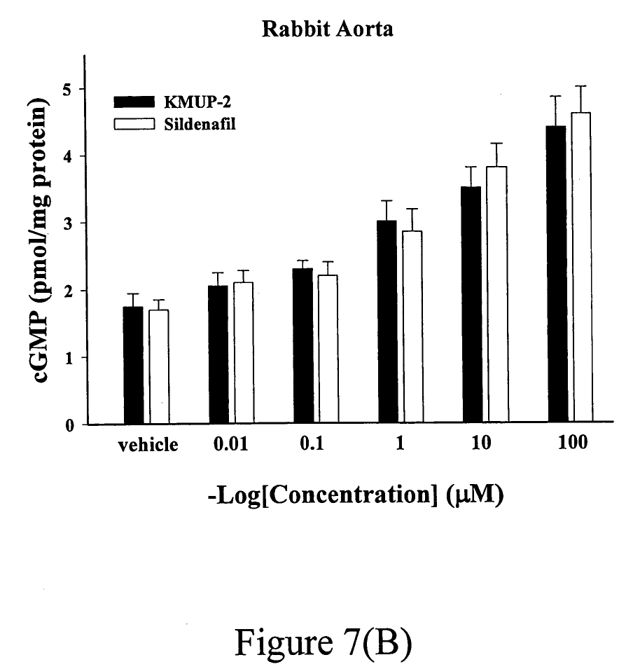

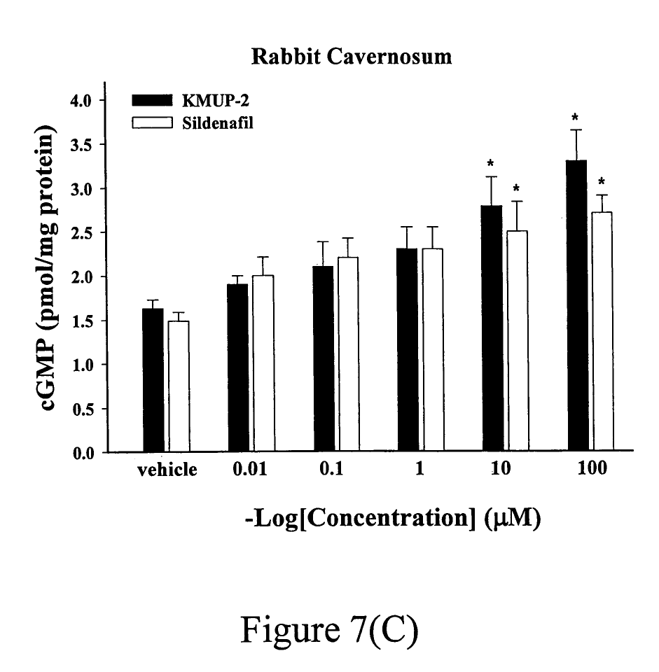

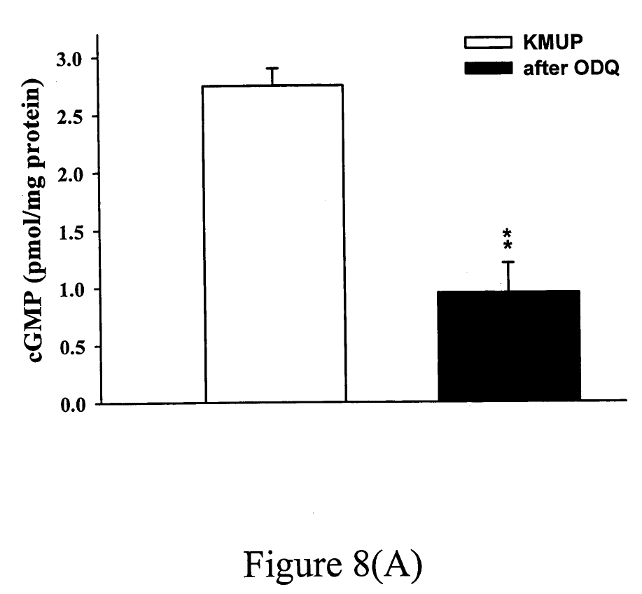

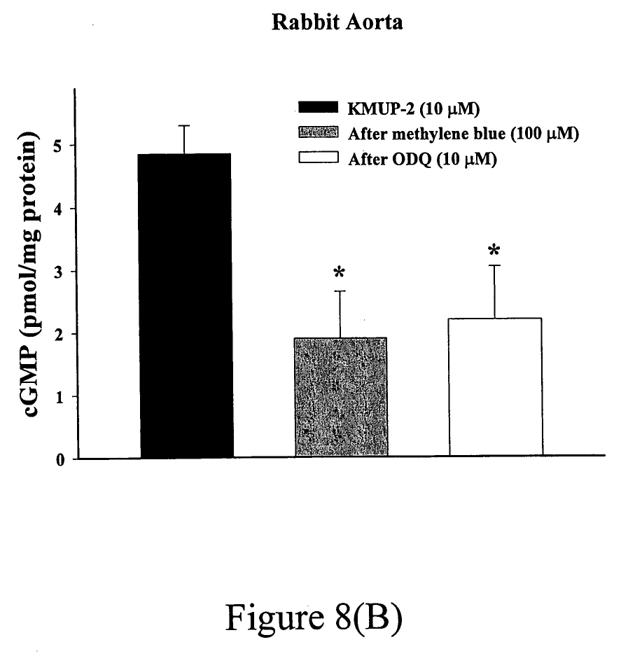

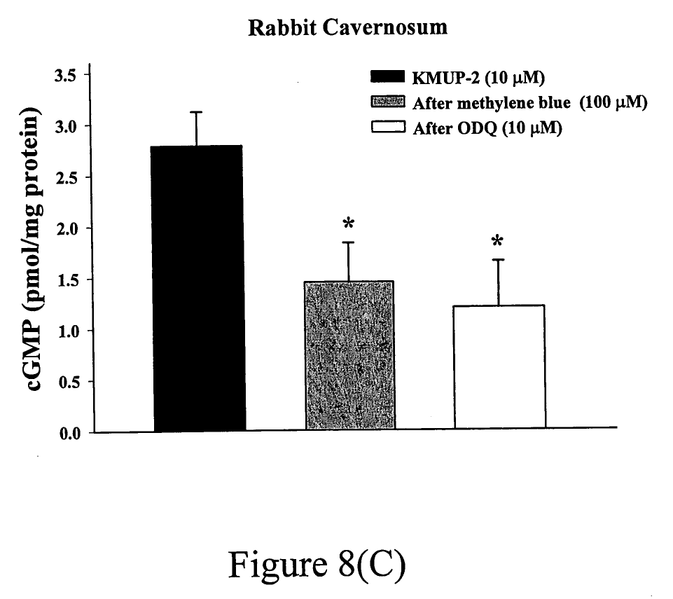

Effects performed by KMUP-1 on guanosine 3′,5′-cyclic monophosphate levels were examined in primary rabbit corpus cavernosum smooth muscle cells in the presence of IBMX (100 μM). The amount of basal release of cGMP was 1.58±0.11 pmol mg⁻¹ well⁻¹ (n=3). KMUP-1 and sildanafil at 0.01-10 μM increased the cGMP levels as shown in FIG. 7A, which were inhibited by the pretreatment with ODQ (10 μM) as shown in FIG. 8A. Furthermore, the effects of KMUP-2 on guanosine 3′,5′-cyclic monophosphate levels in the presence of nonselective PDE (phosphodiesterase) inhibitor IBMX (100 μM) in primary cultured rabbit aorta and corpus cavernosum smooth muscle cells were examined. The amount of basal release of cGMP in aortic and corpus cavernosum smooth muscle cells was 1.65±0.2 and 1.62±0.1 pmol/mg/well (n=3), respectively. KMUP-2 (0.1, 1.0, 10, 100 μM) significantly increased the guanosine 3′,5′-cyclic monophosphate levels in a concentration-dependent manner in rabbit and corpus cavernosum smooth muscle. Sildanafil also elicited significant elevation of cGMP accumulation as shown in FIGS. 7B and 7C. The cGMP levels were significantly inhibited by pretreatments with methylene blue (100 μM) and ODQ (10 μM) in both smooth muscle cells as shown in FIG. 8B.

Increase of ICP (Intracavernous Pressure)

To examine whether KMUP-1 was with corporeal relaxation-associated penile erection activity, the intracavernous pressure of rabbits was measured, and the results are illustrated in Table II.

TABLE 2
Peak increased intracavernous pressure (DICP) and duration
of tumescence respone to KMUP-1 and sildenafil (n = 8)

| | ΔICP (mmHg) | | DT (min) | |
|---|---|---|---|---|
| Doses (mg/kg) | KMUP-1 | Sildenafil | KMUP-1 | Sildenafil |
| 0.2 | 30 ± 5.25 | 28 ± 3.21 | 15 ± 2.24 | 19 ± 3.32 |
| 0.4 | 55 ± 6.21 | 32 ± 4.05 | 27 ± 2.05 | 24 ± 3.45 |
| 0.6 | 67 ± 12.32 | 40 ± 11.05 | 35 ± 3.45 | 28 ± 4.59 |

| | | |
|---|---|---|
| Vehicle | 8 ± 2.11 | 0.4 ± 0.2 |

ICP (mmHg) = intracavernous pressure;
DT = duration of tumescence.
All injection volumes of KMUP-1, sildenafil and vehicle were 0.2 ml. The basal value of ICP recorded was 13.3 ± 2.6 mmHg (n = 6).

As shown in Table 2, the basal value of intracavernous pressure recorded was 13.3±2.6 mmHg (n=6). Intracavemous injection of KMUP-1 induced tumescence as documented by a sustained increase in intracavernous pressure. During the injection periods, the SAP (systemic arterial pressure) and HR (heart rate) were unchanged (data not show). Injection of saline induced a transient rise in intracavernous pressure in a volume-dependent manner. Nevertheless, the pressure rises often returned to the resting level within 1 min and the spike-like pressure curves were different from those of KMUP-1 and sildanafil. It is believed that the transient rise in intracavernous pressure was due to the volume effect of saline. Administration of KMUP-1 and sildanafil (0.2, 0.4, 0.6 mg kg⁻¹ ) induced dose-dependent elevations in intracavernous pressure. It is also found that tumescence of the penile shaft did not show full erection in some cases. There are no significant differences between 2 compounds in their responses to intracavernous pressure as shown in Table 2.

Disruption of Endothelium

The endothelium lining the lacunar spaces of rabbit corpus cavernosum was disrupted and/or removed by detergent treatment using a modification of a protocol for blood vessels, described elsewhere (kim N. et al., J. Clin. Invest., 88, 112-118, 1991). The intact, isolated penis was placed in a tray containing chilled physiological salt solution (PSS). A 21-guage minicatheter was inserted into each corporal body at the proximal end of the penis. A third minicatheter was inserted into the distal end, below the glans penis, where the right and left corpora communicate. While the distal and one proximal minicatheter were clamped, 3 ml of CHAPS (wt vol.⁻¹) in saline was infused into the remaining proximal catheter. After a short interval (˜20 s), the clamped minicatheters were opened and the preparation was washed by infusion of saline. The corpora cavernosa were then removed and tested for endothelial integrity. 75% of the tissues treated with CHAPS did not relax or relaxed poorly (<10% of maximal relaxation) to acetylcholine (Ach, 1 μM) which were considered to be functionally denuded of endothelium.

Tissue Procurement and Organ Bath Experiments

Male rabbits were killed with pentobarbital and their penises excised rapidly and cut longitudinally into equal strips to give 3-6 segments. These segments were incubated in Kerbs-bicarbontate solution (NaCl, 118 mM; NaHCO₃, 25 mM; KCl, 4.7 mM; KH₂PO₄, 1.2 mM; MgSO₄, 1.2 mM; glucose, 11 mM; CaCl₂, 2.5 mM; pH 7.3-7.4) maintained at 37° C. and aerated with 95% O₂ and 5% CO₂. Isometric tension was recorded with a force displacement transducer (UGO BASILE, Model 7004, Italy). Rabbit corpus cavernosum smooth muscles were stretched to a resting tension of 2 g and then contracted with phenylephrine (PE, 10 μM). Tissues were also treated with guanethidine (10 μM) to block contractions caused by noradrenaline, released from adrenergic neuron, and treated with atropine (1 μM) to prevent muscarinic effects caused by acetylcholine. When the stable constriction to phenylephrine was reached, concentration-response curves to KMUP-1 (0.001-10 μM) were constructed. Data were presented as a percentage of the maximum contractile in response to phenylephrine. In order to examine the possible action mechanisms of KMUP-1, the rabbit corpus cavernosum smooth muscles were pretreated with sGC (soluble guanylyl cyclase) inhibitors ODQ (1 μM), a NOS (nitric oxide synthase) inhibitor L-NAME (100 μM), a K⁺ channel blocker Tetraethylammonium (TEA, 10 mM), a K_{ATP} channel blocker glibenclamide (1 μM), a voltage-dependent K⁺ channel blocker 4-AP (100 μM) and Ca²⁺-dpendent K⁺ channel blockers apamin (1 μM) and charybdotoxin (0.1 μM) for 30 min prior to the addition of KMUP-1. In order to observe whether the rabbits corpus cavernosum smooth muscle relaxation of KMUP-1 are affected by a nonselective PDE (phosphodiesterase) inhibitor, the action of KMUP-1 in the presence of IBMX (0.5 μM) is investigated. In another experiment, rabbit corpus cavernosum smooth muscles were preconstricted with 60 mM KCl. The KCl solution was prepared by substituting NaCl with KCl (60 mM) in an equimolar amount.

Culture of Rabbit Corpus Cavernosum Smooth Muscle Cells

Rabbit corpus cavernosum smooth muscles were obtained as sterile surgical specimens, the tissue was washed and cut into 1 to 2 mm pieces and placed into culture dishes with Dulbecco's modified Eagle's medium (DMEM) with 20% fetal bovine serum (FBS), penicillin (100 U ml⁻¹), streptomycin (100 U ml⁻¹) and 2 mM Glutamine. After explants attached to the culture dish, usually 1 to 2 days, DMEM (Dulbecco's modified Eagle's medium) supplement with 10% FBS, penicillin, streptomycin, and glutamine were added. Smooth muscle cells migrated out from the explants in 3-5 days. At this time, the explants were removed, and cells were allowed to achieve confluence. Cells were detached using 0.05% trypsin, 0.02% EDTA at 37° C. for 5 minutes to establish secondary cultures. Cultures were maintained for no more than 4 passages. Cellular homogeneity was further confirmed by the presence of smooth muscle specific a-myosin and a-actin immunoreactivity. Indirect immunofluorescence staining for a variety of antigens was carried out by first plating the cells on chamber slides fixing the cells in 3.7% formaldehyde-phosphate buffered saline for 10 minutes and permeabilizing the cells with phosphate buffered saline 0.1% Triton X-100. Cells were then stained with either a mouse monoclonal antibody directed against the amino terminal 10 amino acids of α-smooth muscle actin or a-myosin (Moreland R. B. et al., J Urol., 153, 826-834, 1995).

Measurement of Intracellular Guanosine 3′,5′-Cyclic Monophosphate Content

Intracellular cGMP concentrations in rabbit corpus cavernosum smooth muscle cells were assayed as our previous disclosure (Wu et al., 2001). In brief, cells were finally grown in 24-well plates (10⁵ cells well⁻¹). At confluence, monolayer cells were washed with PBS and then incubated with KMUP-1 and sildanafil (0.01-10 μM) in the presence of 100 pM IBMX for 10 min as described by Park K. et al. (Biochem Biophys Res Commun., 249, 612-617, 1998). Incubation was terminated by the addition of 6% trichloroacetic acid (TCA). Cell suspensions were sonicated and then centrifuged at 2,500 g for 15 min at 4° C. Then, the supernatants were lyophilized and cGMP of each sample was determined using a commercially available radioimmunoassay kits (Amersham Pharmacia Biotech, Buckinghamshire, England).

Measurement of Intracavemous Pressure

Male rabbits were used for the investigation. After sedation with an intramuscular injection of ketamine (10 mg kg⁻¹), the rabbits were anaesthetized with intraperitoneal pentobarbital (30 mg kg⁻¹) and maintained with 10 mg kg⁻¹ if needed. The animals breathed spontaneously. The rabbits were then placed in the supine position, and the body temperature was maintained at 37° C. The femoral artery was cannulated for continuous monitoring of systemic arterial pressure (SAP) and heart rate (HR) via a pressure transducer (Spectramed, Model P10EZ, U.S.A.). Under sterile conditions, the skin overlying the penis was incised and the corpora cavernosum was exposed at the root of the penis. A 25-gunge needle was inserted into the corpus cavernosum smooth muscles for pressure recording. The needle was connected to a three-way stopcock, thus permitting the intracavernous injection of the drugs. The tube was filled with heparinized saline (50 IU 2h⁻¹) to prevent clotting.

Intracavemous Injection

In 48 rabbits, divided into 2 groups, 8 animals in each group for one dose, KMUP-1 and sildanafil (0.2, 0.4, 0.6 mg kg⁻¹) were, respectively, injected into corpus cavernosum smooth muscles in a volume of less than 0.2 ml. Normal saline in increasing volumes (0.05, 0.1, 0.2 ml) was injected in four rabbits as a control group. The effects of KMUP-1 and normal saline on the intracavernous pressure (ICP) and on the duration of action were evaluated. In order to minimize the effect of the previous drug, the cavernous body was flushed with 0.2 ml normal saline before each injection and the time interval between each injection was at least 1.5 h.

Statistical Evaluation of Data

The results are expressed as mean ± s.e.mean. Statistical differences were determined by independent and paired Student's t-test in unpaired and paired samples, respectively. Whenever a control group was compared with more than one treated group, the one way ANOVA or two way repeated measures ANOVA was used. When the ANOVA manifested a statistical difference, the Dunnett's or Student-Newman-Keuls test was applied. The P value less than 0.05 was considered to be significant in all experiments. Analysis of the data and plotting of the figures were done with the aid of software (SigmaStat and SigmaPlot, Version 5.0, San Rafael, Calif., U.S.A.) run on an IBM compatible computer.

### EXAMPLE 1

### Synthesis of Compound A (N7-Bromoethyl Theophylline)

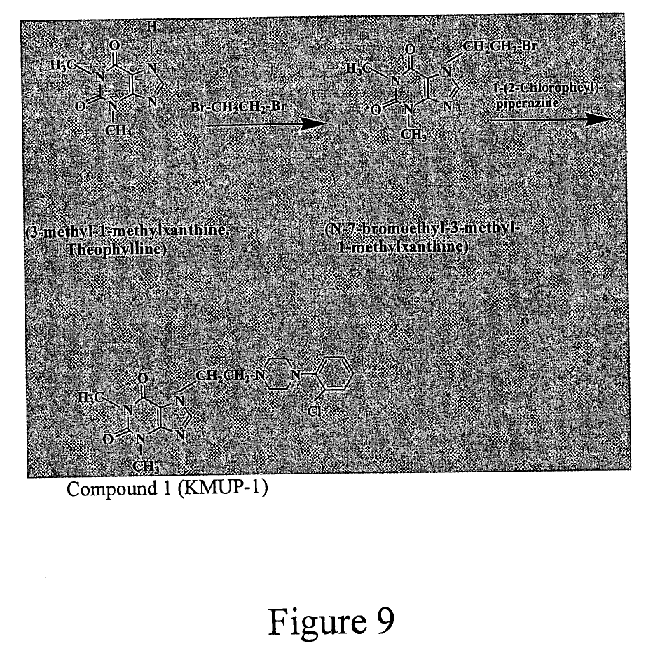

0.2 mole theophylline (1,3-dimethylxanthine) was dissolved into 0.4 mole dibromoethane in a glass reacter, then boiled and mixed at 100° C. on mantle heater and refluxed for 4 hours, equipped with a cooling condenser to return the vaporized solvent. Till solid completely melt, the reaction was added with a catalyst, 125 ml 1.6 N NaOH, to react under 100° C. and be boiled for overnight, so as to form white solid precipitated and NaBr soluble in upper water layer. The white solid precipitate was filtered, dissolved in methanol, and concentrated under reduced pressure to obtain white coarse crystal, and then recrystallized with methanol to obtain the pure white crystal compound A (N7-bromoethyl-3 methyl-1-methylxanthine, N7-bromoethyl theophylline. Please refer to FIG. 9 showing the synthesis scheme of the compound A.

### EXAMPLE 2

### Synthesis of Compound B (N7-Bromoethyl IBMX)

0.2 mole 3-isobutyl-1-methylxanthine (IBMX) was dissolved into 0.4 mole dibromoethane in a glass reacter, then boiled and mixed at 100° C. on mantle heater, refluxed for 4 hours, and equipped with a cooling condenser to return the vaporized solvent. Till solid completely melt, then the reaction was added with a catalyst, 125 ml 1.6 N NaOH, to react under 100° C. and be boiled for overnight, so as to form white solid precipitated and NaBr soluble in upper water layer. The white solid precipitate was filtered, dissolved in methanol, concentrated under reduced pressure to obtain white coarse crystal, and then recrystallized with methanol to obtain the pure white crystal compound B (N7-bromoethyl IBMX).

### EXAMPLE 3

### Synthesis of Theophylline-Based Derivatives (KMUPs) from Compound A

The compound A dissolved in methanol, according to targeting products required, was added with 1-(2-Chlorophenyl)piperazine (in compound 1), 1-(m-Chlorophenyl) (in compound 2 piperazine, 1-(4-Nitrophenyl) piperazine (in compound 4), and 1-(o-Metyhoxyphenyl) piperazine (in compound 8), respectively, to process the amination, using NaOH as catalyst, and reflux for 4 hours in methanol to precipitate , to dissolve the solid product, and to recrystallize with methanol to obtain compounds.

### EXAMPLE 4

### Synthesis of Theophylline-Based Compound from Compound B

According to the example 3, the compound A (N7-bromoethyl theophylline) was replaced with the compound B (N7-bromoethyl IBMX) to obtain derivative compounds.

Furthermore, in accordance with the present invention, a tablet including KMUPs (KMUP-1 analogues) is provided, in which the tablet includes 50 mg of KMUP-1 analogues, 30 mg of lactose, 4 mg of starch, 6 mg of magnesium stearate and 10 mg of corn starch.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

1. A pharmaceutical composition, comprising:
7-[2-[4-(2-methoxyphenyl)piperazinyl]-ethyl]-1,3-dimethylxanthine (KMUP-1); and
a pharmaceutically acceptable carrier.

2. A pharmaceutical composition, comprising:
7-[2-[4-(2-chlorophenyl)piperazinyl]-ethyl]-1,3-dimethylxanthine (KMUP-1); and
a pharmaceutically acceptable carrier.

3. A compound of the formula (I) synthesized from one of theophylline (1-methyl, 3-methyl xanthine) and IBMX (1-methyl-3-isobutyl xanthine): 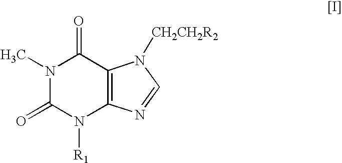
wherein R₁ is one of —CH₃ and CH₂CH(CH₃)₂, and
R₂ is 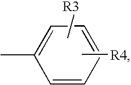
in which R₃ and R₄ are independently selected from a group consisting of OCH₃, CH₃ and NO₂.

4. A compound of the formula (II) synthesized from one of theophylline (1-methyl, 3-methyl xanthine) and IBMX (1-methyl-3-isobutyl xanthine): 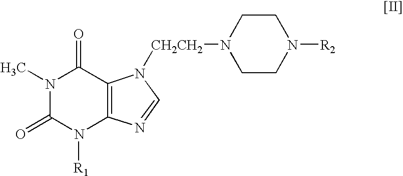
wherein R₁ is one of —CH₃ and CH₂CH(CH₃)₂, and
R₂ is 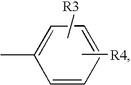
10 in which R₃ and R₄ are independently selected from a group consisting of OCH₃, CH₃ and NO₂.
